SEQUENCE LISTING (29 sequences)

SEQ ID NO: 1 - protein, 168 aa, synthetic construct
MESNQSNNGGSGNAALNRGGRYVPPHLRGGDGGAAAAASAGGDDRRGGAGGGGYRRGGGNSGGGGGGGYDRGYNDNRDDRDNRGGSGGYGRDRNYEDRGYNGGGGGGGNRGYNNNRGGGGGGYNRQDRGDGGSSNFSRGGYNNRDEGSDNRGSGRSYNNDRRDNGGDG

SEQ ID NO: 2 - protein, 196 aa, synthetic construct
MESNQSNNGGSGNAALNRGGRYVPPHLRGGDGGAAAAASAGGDDRRGGAGGGGYRRGGGNSGGGGGGGYDRGYNDNRDDRDNRGGSGGYGRDRNYEDRGYNGGGGGGGNRGYNNNRGGGGGGYNRQDRGDGGSSNFSRGGYNNRDEGSDNRGSGRSYNNDRRDNGGDGENLYFQGMSKGPWGRGRGRGWPGVGYGY

SEQ ID NO: 3 - protein, 210 aa, synthetic construct
MESNQSNNGGSGNAALNRGGRYVPPHLRGGDGGAAAAASAGGDDRRGGAGGGGYRRGGGNSGGGGGGGYDRGYNDNRDDRDNRGGSGGYGRDRNYEDRGYNGGGGGGGNRGYNNNRGGGGGGYNRQDRGDGGSSNFSRGGYNNRDEGSDNRGSGRSYNNDRRDNGGDGENLYFQGMSKGPWGRGRGRGWPGVGYWGRGRGRGWPGVGYGY

SEQ ID NO: 4 - protein, 7 aa, synthetic construct
ENLYFQG

SEQ ID NO: 5 - protein, 236 aa, synthetic construct
GESLFKGPRDYNPISSTICHLTNESDGHTTSLYGIGFGPFIITNKHLFRRNNGTLVVQSLHGVFKVKNTTTLQQHLIDGRDMIIIRMPKDFPPFPQKLKFREPQREERICLVTTNFQTKSMSSMVSDTSCTFPSGDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMN

SEQ ID NO: 6 - protein, 7 aa, synthetic construct
ENLYFQS

SEQ ID NO: 7 - protein, 14 aa, synthetic construct
WGRGRGRGWPGVGY

SEQ ID NO: 8 - DNA, 31 bp, synthetic construct
  source 1..31: mol_type = other DNA
atggaatcaaatcaatcaaataatggcggaa

SEQ ID NO: 9 - DNA, 38 bp, synthetic construct
  source 1..38: mol_type = other DNA
gctgccgctgccgctaccatagccgtagccgactccag

SEQ ID NO: 10 - DNA, 50 bp, synthetic construct
  source 1..50: mol_type = other DNA
attatttgattgatttgattccatggccgcgctagttctagaaaacttag

SEQ ID NO: 11 - DNA, 46 bp, synthetic construct
  source 1..46: mol_type = other DNA
ggtagcggcagcggcagcatgagcaaaggagaagaacttttcactg

SEQ ID NO: 12 - DNA, 31 bp, synthetic construct; identical to SEQ ID NO: 8
  source 1..31: mol_type = other DNA

SEQ ID NO: 13 - DNA, 49 bp, synthetic construct
  source 1..49: mol_type = other DNA
tacacatggcatggatgagctctacaaacaccaccaccaccaccactga

SEQ ID NO: 14 - DNA, 34 bp, synthetic construct
  source 1..34: mol_type = other DNA
ccaccaccactgagatccggctgctaacaaagcc

SEQ ID NO: 15 - DNA, 47 bp, synthetic construct
  source 1..47: mol_type = other DNA
ttaactttaagaaggagatataccatatggaatcaaatcaatcaaat

SEQ ID NO: 16 - DNA, 52 bp, synthetic construct
  source 1..52: mol_type = other DNA
gtcaaggagaaaaaactataggtgaatctttatttaagggtcctcgtgacta

SEQ ID NO: 17 - DNA, 59 bp, synthetic construct
  source 1..59: mol_type = other DNA
agtcaaggaagccactcaattgatgaacgggctattgatcatgtaattagttatgtcac

SEQ ID NO: 18 - DNA, 50 bp, synthetic construct
  source 1..50: mol_type = other DNA
tggcactggccgtcgttttaacatggcattaccaccatatacatatccat

SEQ ID NO: 19 - DNA, 59 bp, synthetic construct
  source 1..59: mol_type = other DNA
aattgttaatatacctctatactttaacgtcaaggagaaaaaactataggtgaatcttt

SEQ ID NO: 20 - DNA, 59 bp, synthetic construct
  source 1..59: mol_type = other DNA
actcaattgatgaacgggctattgatcatgtaattagttatgtcacgcttacattcacg

SEQ ID NO: 21 - DNA, 51 bp, synthetic construct
  source 1..51: mol_type = other DNA
cgtgcgtattatctcttaactcataatgccggccgcaaattaaagccttcg

SEQ ID NO: 22 - DNA, 28 bp, synthetic construct
  source 1..28: mol_type = other DNA
ttggtaacagaagatggcagtatttcca

SEQ ID NO: 23 - DNA, 50 bp, synthetic construct
  source 1..50: mol_type = other DNA
gcgtaatcatggtcatagctgtttcctgggagatgcgacgaattactggc

SEQ ID NO: 24 - DNA, 50 bp, synthetic construct
  source 1..50: mol_type = other DNA
gaaggctttaatttgcggccggcattatgagttaagagataatacgcacg

SEQ ID NO: 25 - DNA, 30 bp, synthetic construct
  source 1..30: mol_type = other DNA
gagaaaggacttaatccgtacacaatgatt

SEQ ID NO: 26 - DNA, 50 bp, synthetic construct
  source 1..50: mol_type = other DNA
gccagtaattcgtcgcatctcccaggaaacagctatgaccatgattacgc

SEQ ID NO: 27 - DNA, 50 bp, synthetic construct
  source 1..50: mol_type = other DNA
atggatatgtatatggtggtaatgccatgttaaaacgacggccagtgcca

SEQ ID NO: 28 - DNA, 22 bp, synthetic construct
  source 1..22: mol_type = other DNA
aacaatgggtttctggctggag

SEQ ID NO: 29 - DNA, 34 bp, synthetic construct
  source 1..34: mol_type = other DNA
ccatcaatggcttctaaaagttttcaaagaagtg

(19) United States
(12) Patent Application Publication
(10) Pub. No.: US 2026/0117169 A1
(43) Pub. Date: Apr. 30, 2026

(54) METHOD FOR FORMING REVERSIBLE MEMBRANELESS ORGANELLE IN MICROORGANISMS

(71) Applicants: JITRI FUTURE FOOD TECHNOLOGY INSTITUTE CO., LTD., Yixing, Wuxi (CN); JIANGNAN UNIVERSITY, Wuxi (CN)

(72) Inventors: Long LIU, Wuxi (CN); Jian CHEN, Wuxi (CN); Xueqin LV, Wuxi (CN); Guocheng DU, Wuxi (CN); Ke JIN, Wuxi (CN); Ziyang HUANG, Wuxi (CN)

(21) Appl. No.: 19/006,000

(22) Filed: Dec. 30, 2024

Related U.S. Application Data
(63) Continuation of application No. PCT/CN2024/088179, filed on Apr. 17, 2024.

(30) Foreign Application Priority Data
Oct. 20, 2023 (CN) 202311365367.9

(51) Int. Cl. C12N 1/18; C12N 15/81; C12P 21/02; C12R 1/865

(52) U.S. Cl. CPC C12N 1/18; C12N 15/81; C12P 21/02; C12R 2001/865

(57) ABSTRACT

The present invention provides a method for forming a reversible membraneless organelle in microorganisms. In the present invention, in order to form a membraneless organelle in a host cell, an artificial short peptide sequence WGR-1 is fused on the basis of an intrinsically disordered protein RGG. Fusion of one WGR-1 sequence or two WGR-1 sequences connected in series can both effectively promote phase separation of RGG, thus forming a membraneless organelle in Saccharomyces cerevisiae, wherein the membraneless organelle RGG-(WGR-1)₂ has a better liquid fluidity. RGG-x-(WGR-1)₂ is further constructed by introducing an enzyme cleavage site x into the RGG-(WGR-1)₂ system in the present invention, and the reversible formation of the membraneless organelle is achieved after expression of a protease is induced. The method of the present invention has important value in customizing and controlling cell functions and the like.

This application is a Continuation application of PCT/CN2024/088179, filed on Apr. 17, 2024, which claims priority to Chinese Patent Application No. 202311365367.9, filed on Oct. 20, 2023, which is incorporated by reference for all purposes as if fully set forth herein.

A Sequence Listing XML file named “10015_0166.xml” created on Dec. 30, 2024, and having a size of 27,557 bytes, is filed concurrently with the specification. The sequence listing contained in the XML file is part of the specification and is herein incorporated by reference in its entirety.

### FIELD OF THE INVENTION

The present invention relates to a method for forming a reversible membraneless organelle in microorganisms, belonging to the field of biotechnology.

### DESCRIPTION OF THE RELATED ART

Enhancing the synthesis efficiency of microbial cell factory products through organelle engineering is a commonly used synthetic biological means currently, mainly focusing on modification of endogenous organelles and construction of artificial organelles. Among them, the modification of endogenous organelles has problems, such as it is difficult for substances to cross the membrane, and it is difficult to control the metabolic network quickly and reversibly because endogenous organelles cannot be assembled or disassembled. The membraneless organelles formed by liquid-liquid phase separation based on intrinsically disordered proteins effectively avoid the above problems. Membraneless organelles with dynamic liquid fluidity can act as chemical reactors in cells, achieve limited permeation of substances, and achieve reversible phase separation of membraneless organelles by responding to chemical signals.

It is difficult for a single intrinsically disordered protein RGG to form a membraneless organelle in Saccharomyces cerevisiae. By expressing multiple RGGs connected in series, RGG phase separation can be promoted and a membraneless organelle can be formed in Saccharomyces cerevisiae. Or by expressing reported intrinsically disordered proteins such as FUSN and A-IDPs in Saccharomyces cerevisiae to form membraneless organelles. The intrinsically disordered proteins such as RGG (168 amino acids), FUSN (216 amino acids) and A-IDPs (175 amino acids) themselves have long sequences. Connection of multiple intrinsically disordered proteins in series repeatedly can effectively enhance their phase separation ability, but the construction process of connection them in series repeatedly is difficult because of the long length of these intrinsic proteins. Therefore, it is necessary to find a new method for modification of endogenous organelles.

### SUMMARY OF THE INVENTION

In order to solve the above problems, in the present invention, the protein sequence in the process of forming a membraneless organelle is modified to enhance the phase separation effect, and a membraneless organelle with a high liquid fluidity is obtained. On this basis, a construction method of an intracellular reversible membraneless organelle is developed to achieve the reversible formation of the membraneless organelle in different host cells.

The first object of the present invention is to provide a method for forming a membraneless organelle in microorganisms, including the following steps:
introducing a protein sequence for forming the membraneless organelle into a host cell, wherein the protein sequence for forming the membraneless organelle includes an intrinsically disordered protein with an amino acid sequence as shown in SEQ ID NO: 1, and a short peptide located downstream of the intrinsically disordered protein, wherein the short peptide is a sequence as shown in SEQ ID NO: 7 or two SEQ ID NO: 7 sequences connected in series (two repeat sequences are connected directly).

Preferably, the microorganism in the present invention can be any host cell that needs to synthesize proteins in living cells, including but not limited to Enterobacterium (such as E. coli), yeast (such as Saccharomyces cerevisiae) and the like.

The second object of the present invention is to provide a method for forming a reversible membraneless organelle in microorganisms, including the following steps:
introducing a protein sequence containing an enzyme cleavage site for forming the membraneless organelle and a gene sequence encoding a cleavage enzyme into a host cell, wherein
the protein sequence containing the enzyme cleavage site for forming the membraneless organelle includes an intrinsically disordered protein with an amino acid sequence as shown in SEQ ID NO: 1, a short peptide located downstream of the intrinsically disordered protein, and the enzyme cleavage site between the intrinsically disordered protein and the short peptide, wherein the short peptide is the sequence shown in SEQ ID NO: 7 or two SEQ ID NO: 7 sequences connected in series; and
cleaving the enzyme cleavage site specifically by the cleavage enzyme.

Preferably, the gene sequence encoding the cleavage enzyme is regulated by a first promoter, and the protein sequence containing the enzyme cleavage site for forming the membraneless organelle is regulated by a second promoter, and the first promoter is an inducible promoter and is different from the second promoter, and the second promoter can be an inducible promoter or a constitutive promoter.

Preferably, the cleavage enzyme and the cleavage site can be arbitrarily selected as required, as long as specific cleavage can be achieved in living cells. For example, in the present invention, the TEV protease (the amino acid sequence is shown in SEQ ID NO: 5) is selected as the cleavage enzyme, and the cleavage site of the TEV enzyme is between glutamine and glycine/serine, so the sequence of the enzyme cleavage site can be SEQ ID NO: 4 or SEQ ID NO: 6.

More preferably, the protein sequence containing the enzyme cleavage site for forming the membraneless organelle is shown in SEQ ID NO: 2 or SEQ ID NO: 3. Specifically,

SEQ ID NO: 2: amino acid sequence of RGG-(WGR-1)₁
MESNQSNNGGSGNAALNRGGRYVPPHLRGGDGGAAAAASAGGDDRRGGAG
GGGYRRGGGNSGGGGGGGYDRGYNDNRDDRDNRGGSGGYGRDRNYEDRGY
NGGGGGGGNRGYNNNRGGGGGGYNRQDRGDGGSSNFSRGGYNNRDEGSDN
RGSGRSYNNDRRDNGGDGENLYFQGMSKGPWGRGRGRGWPGVGYGY
SEQ ID NO: 3: amino acid sequence of RGG-(WGR-1)₂
MESNQSNNGGSGNAALNRGGRYVPPHLRGGDGGAAAAASAGGDDRRGGAG
GGGYRRGGGNSGGGGGGGYDRGYNDNRDDRDNRGGSGGYGRDRNYEDRGY
NGGGGGGGNRGYNNNRGGGGGGYNRQDRGDGGSSNFSRGGYNNRDEGSDN
RGSGRSYNNDRRDNGGDGENLYFQGMSKGPWGRGRGRGWPGVGYWGRGRG
RGWPGVGYGY

The third object of the present invention is to provide a recombinant protein for constructing a membraneless organelles, including an intrinsically disordered protein with an amino acid sequence as shown in SEQ ID NO: 1, and a short peptide located downstream of the intrinsically disordered protein, wherein the short peptide is a sequence as shown in SEQ ID NO: 7 or two SEQ ID NO: 7 sequences connected in series.

The fourth object of the present invention is to provide a recombinant bacterium containing a membraneless organelle forming protein, containing the recombinant protein above.

The fifth object of the present invention is to provide a system for constructing a reversible membraneless organelle, including two parts: a protein sequence containing an enzyme cleavage site for forming a membraneless organelle and a gene sequence for coding a cleavage enzyme, wherein
the protein sequence containing the enzyme cleavage site for forming the membraneless organelle includes an intrinsically disordered protein with an amino acid sequence as shown in SEQ ID NO: 1, a short peptide located downstream of the intrinsically disordered protein, and the enzyme cleavage site between the intrinsically disordered protein and the short peptide, wherein the short peptide is the sequence shown in SEQ ID NO: 7 or two SEQ ID NO: 7 sequences connected in series; and
the cleavage enzyme specifically cleaves the enzyme cleavage site.

Preferably, plasmids can be used as vectors for the above two parts, respectively.

The sixth object of the present invention is to provide a recombinant bacterium containing a reversible membraneless organelle, containing the system above.

The seventh object of the present invention is to provide a method for producing a protein in living cells, including the following steps:
introducing the recombinant protein or the system above into the living cells.

Preferably, the living cells are microbial cells or animal and plant cells ex-vivo (even stem cells are commercial stem cells obtained after being approved by ethical review).

The eighth object of the present invention is to provide use of the recombinant protein, the system or the recombinant bacterium in preparing cell drugs or protein drugs. Specifically, they are used for protein customization and modification in a pharmaceutical process.

The present invention has the following beneficial effects:
(1) In the present invention, after artificial sequences WGR-1 connected in series are further expressed on the basis of an intrinsically disordered protein RGG, the phase separation ability of RGG is effectively enhanced, and finally a membraneless organelle RGG-(WGR-1)₂ with a high liquid fluidity is obtained. In vitro, the fluidity of RGG-(WGR-1)₂ is as high as 89%. Compared with existing long repeat sequences connecting a plurality of intrinsically disordered proteins in series, the membraneless organelle system of the present invention is easier to construct.
(2) RGG-x-(WGR-1)ₙ is further constructed by introducing an enzyme cleavage site x into the membraneless organelle RGG-(WGR-1)₂ in the present invention, and the reversible formation of the membraneless organelle is successfully achieved after expression of a cleavage enzyme is induced, thus providing a convenient and rapid regulation strategy for the membraneless organelle.

### BRIEF DESCRIPTION OF THE DRAWINGS

FIG. 1 is the verification result of the membraneless organelle RGG-x-(WGR-1)ₙ-EGFP in vivo.

FIG. 2 is the verification result of the RGG-x-(WGR-1)ₙ-EGFP membraneless organelle in vitro.

FIG. 3 is the reversible formation result of the membraneless organelle system RGG-x-(WGR-1)₂-EGFP induced by Saccharomyces cerevisiae.

### DETAILED DESCRIPTION OF THE PREFERRED EMBODIMENTS

The present invention will be further described with the attached drawings and specific examples, so that those skilled in the art can better understand and implement the present invention, but the examples given are not taken as limitations of the present invention.

The technical solutions involved in the present invention is as follows:

In the present invention, yeast was used as a starting strain, and the effect of an artificial short peptide sequence WGR-1 (SEQ ID NO: 7) with a length of only 14 amino acids on RGG phase separation was investigated, so as to construct an RGG-(WGR-1)ₙ-EGFP membraneless organelle (n represents the number of repeats);
in addition, in the present invention, a cleavage site x was introduced into the phase separation system formed by RGG-(WGR-1)ₙ-EGFP, and an RGG-x-(WGR-1)ₙ-EGFP controllable membraneless organelle system was constructed by inducing the expression of an enzyme that specifically cleaved the site x.

In the present invention, the principle of forming a reversible membraneless organelle is as follows: liquid-liquid phase separation mediated by multivalent interaction is the basis of forming a membraneless organelle. The introduction of enzyme cleavage sites of proteins can change multivalent interactions and achieve the reversible formation of membraneless organelles. Therefore, after an enzyme cleavage site and a corresponding enzyme thereof are introduced into a host cell at the same time, and before the expression of the enzyme is induced, WGR-1 can promote the phase separation of RGG, so as to form a membraneless organelle; after the expression of the enzyme is induced and the cleavage site is cleaved, the RGG protein is disconnected from the WGR-1 short peptide to destroy the phase separation system formed by RGG-(WGR-1)ₙ, and the membraneless organelle disappears. In this way, the controllable construction of the membraneless organelle can be achieved.

### Example 1: Construction of Membraneless Organelle of Saccharomyces cerevisiae Based on Artificial Sequence RGG-x-(WGR-1)ₙ-EGFP

a) Gene fragments RGG (amino acid sequence SEQ ID NO.1), RGG-x-(WGR-1) (amino acid sequence SEQ ID NO: 2) and RGG-x-(WGR-1)₂ (amino acid sequence SEQ ID NO: 3) were synthesized artificially, where x is an enzyme cleavage site specifically recognized by a TEV protease (amino acid sequence SEQ ID NO: 4), and the amino acid sequences 1, 2 and 3 were amplified by primers RG-F (gene sequence SEQ ID NO: 8) and RG-R (gene sequence SEQ ID NO: 9) respectively to obtain target gene fragments RG, RG1 and RG2.
b) A vector fragment pESC containing a promoter TEF1, a green fluorescent protein EGFP and a terminator CYC1 was obtained by using a plasmid pESC-TEF1-EGFP-CYC1 as a template and using primers pESC-F (gene sequence SEQ ID NO: 10) and pESC-R (gene sequence SEQ ID NO: 11) for amplification.
c) The target gene fragments RG, RG1 and RG2 were fused and expressed with pESC respectively to obtain plasmids pESC-TEF1-RGG-EGFP-CYC1, pESC-TEF1-RGG-(WGR-1)₁-EGFP-CYC1 and pESC-TEF1-RGG-(WGR-1)₂-EGFP-CYC1.

```
Primer sequence
RG-F:
  (SEQ ID NO: 8)
ATGGAATCAAATCAATCAAATAATGGCGGAA
RG-R:
  (SEQ ID NO: 9)
GCTGCCGCTGCCGCTACCATAGCCGTAGCCGACTCCAG
pESC-F:
  (SEQ ID NO: 10)
ATTATTTGATTGATTTGATTCCATGGCCGCGCTAGTTCTAGAAAACTTAG
pESC-R:
(SEQ ID NO: 11)
GGTAGCGGCAGCGGCAGCATGAGCAAAGGAGAAGAACTTTTCACTG
```

### Example 2: Verification of Membraneless Organelle RGG-x-(WGR-1)ₙ-EGFP In Vivo

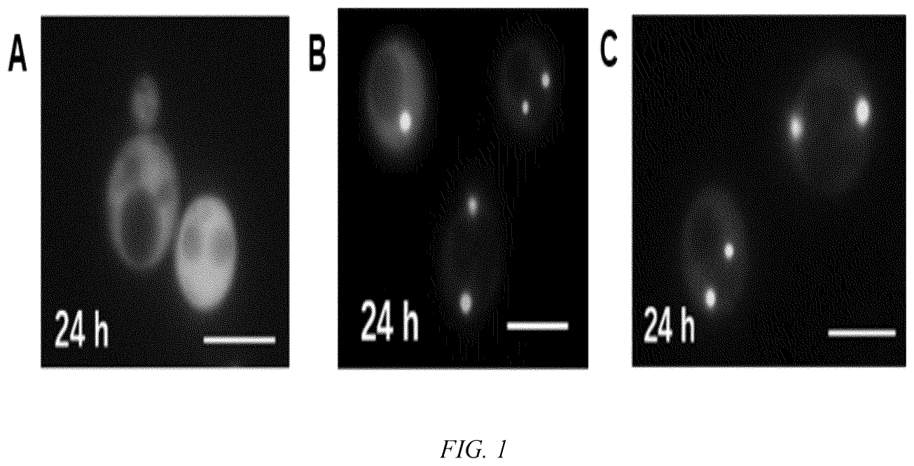

a) The plasmids pESC-TEF1-RGG-EGFP-CYC1, pESC-TEF1-RGG-(WGR-1)₁-EGFP-CYC1 and pESC-TEF1-RGG-(WGR-1)₂-EGFP-CYC1 were respectively transformed into Saccharomyces cerevisiae S288C, coated on G418 resistant plates and cultured at 30° C. for 2 days to obtain strains R-EGFP, RW1-EGFP and RW2-EGFP.
b) Single colonies of R-EGFP, RW1-EGFP and RW2-EGFP were picked up respectively and cultured in a 2 mL of YPD liquid medium for 24 h.
c) R-EGFP, RW1-EGFP and RW2-EGFP were observed with a fluorescence microscope. As shown in FIG. 1-A, RGG cannot form aggregates in Saccharomyces cerevisiae. As shown in FIGS. 1-B and 1-C, RW1-EGFP and RW2-EGFP all form spherical aggregates in cells (all the length of scales in FIG. 1-3 is 5 um).

### Example 3: Verification of Membraneless Organelle Based on Artificial Sequence RGG-x-(WGR-1)ₙ-EGFP In Vitro

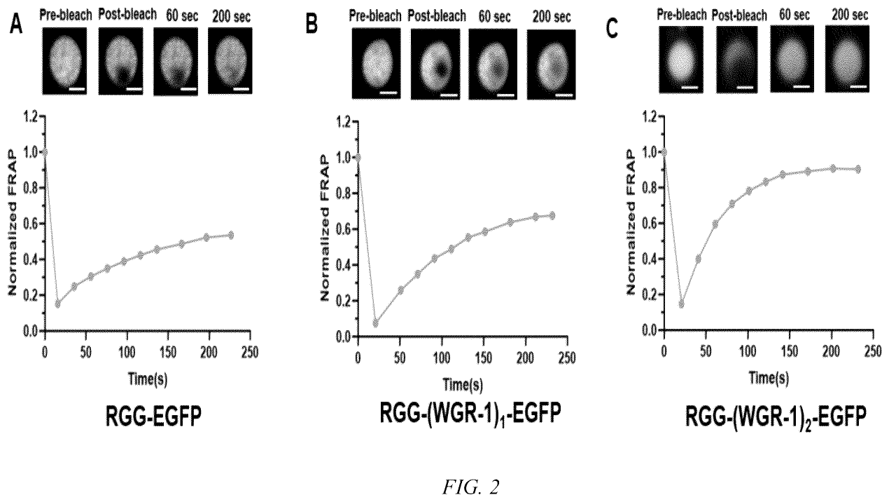

a) Target gene fragments ER, ERG1 and ERG2 were obtained by using primers ERG-F (gene sequence SEQ ID NO: 12) and ERG-R (gene sequence SEQ ID NO: 13) to amplify the amino acid sequences 1, 2 and 3.
b) A vector fragment pET containing a promoter T7 and a 6×His tag was obtained by using a plasmid pET28a-T7-His as a template and using primers pET-F (gene sequence SEQ ID NO: 14) and pET-R (gene sequence SEQ ID NO: 15) for amplification.
c) The target gene fragments ER, ERG1 and ERG2 were fused and expressed with pET respectively to obtain plasmids pET-RGG, pET-RGG-(WGR-1)₁-EGFP-His and pET-RGG-(WGR-1)₂-EGFP-His.
d) The plasmids pET-RGG, pET-RGG-(WGR-1)₁-EGFP and pET-RGG-(WGR-1)₂-EGFP were respectively transformed into E. coli BL21(DE3), coated on Kana resistant plates and cultured overnight at 37° C. to obtain strains ER-EGFP-His, ERW1-EGFP-His and EWR2-EGFP-His.
e) Single colonies of ER-EGFP-His, ERW1-EGFP-His and EWR2-EGFP-His were picked up, cultured in a 2 mL of LB liquid medium for 8-10 h, and then transferred to a TB medium at 1-5%. When OD₆₀₀ was 0.6-1, IPTG was added with a final concentration of 0.5 mM, and cultured at 25° C. for 24 h.
f) The fermentation broth was purified by using a nickel column. Buffer A was 40 mM Tris-HCl (40 mM, pH7.4) with pH=7.4. Buffer B was 500 mM imidazole solution prepared from Solution A. Linear elution (0-100% Buffer B) was used to collect the target samples RGG-EGFP, RGG-(WGR-1)₁-EGFP and RGG-(WGR-1)₂-EGFP.
g) Fluorescence recovery after photobleaching can be used to analyze the fluidity of a protein condensate. The purified target sample was placed under a laser confocal microscope to carry out an experiment of fluorescence recovery after photobleaching, and the results are shown in FIG. 2. The fluorescence inside RGG-EGFP drops can be recovered to 54% of that before photobleaching (FIG. 2-A), RGG-(WGR-1)₁-EGFP can reach 65%, and RGG-(WGR-1)₂-EGFP is the highest, reaching 89%, indicating that RGG-(WGR-1)₂-EGFP drops have a fluidity inside the drops, and have the best fluidity.

```
ERG-F:
  (SEQ ID NO: 12)
ATGGAATCAAATCAATCAAATAATGGCGGAA
ERG-R:
(SEQ ID NO: 13)
TACACATGGCATGGATGAGCTCTACAAACACCACCACCACCACCACTGA
pET-F:
  (SEQ ID NO: 14)
CCACCACCACTGAGATCCGGCTGCTAACAAAGCC
pET-R:
  (SEQ ID NO: 15)
TTAACTTTAAGAAGGAGATATACCATATGGAATCAAATCAATCAAAT
```

### Example 4: Construction of Reversible Membraneless Organelle

a) A gene fragment TEV protease (amino acid sequence SEQ ID NO: 5) was artificially synthesized and amplified by using primers TEV-F (gene sequence SEQ ID NO: 16) and TEV-R (gene sequence SEQ ID NO: 17) to obtain a target gene fragment TEV.
b) A promoter gene fragment GAL was obtained by using the genome of Saccharomyces cerevisiae S288C as a template, and using primers GAL1-F (gene sequence SEQ ID NO: 18) and GAL1-R (gene sequence SEQ ID NO: 19) for amplification; a terminator gene fragment CYC1 was obtained by amplification using CYC1-F (sequence SEQ ID NO: 20) and CYC1-R (sequence SEQ ID NO: 21) primers; a target gene fragment 1021b-UP was amplified by using primers UP-F (sequence SEQ ID NO: 22) and UP-R (sequence SEQ ID NO: 23); the target gene fragment 1021b-DOWN was amplified by using primers DOWN-F (sequence SEQ ID NO: 24) and DOWN-R (sequence SEQ ID NO: 25).
c) A gene fragment LOXP-LEU was artificially synthesized and amplified by using primers LOXP-F (gene sequence SEQ ID NO: 26) and LOXP-R (gene sequence SEQ ID NO: 27) to obtain a target gene fragment LOXP.
d) The gene fragments TEV, 1021b-UP, 1021b-DOWN and LOXP were subjected to a fusion PCR by PCR, and the correct bands obtained by gel running were recovered by a column to obtain a fusion gene fragment 1021b-GAL1-TEV-CYC1.
e) The fusion gene fragment 1021b-GAL1-TEV-CYC1 was transformed into the strain RW2-EGFP obtained in Example 2, coated on an SD-His plate, and cultured at 30° C. for 2 days. The colonies were subjected to PCR with primers YZ-TEV-F (gene sequence SEQ ID NO: 28) and YZ-TEV-R (gene sequence SEQ ID NO: 29), and the resulting strain TEV-RW2-EGFP was verified.

```
TEV-F:
(SEQ ID NO: 16)
GTCAAGGAGAAAAAACTATAGGTGAATCTTTATTTAAGGGTCCTCGTGAC
TA
TEV-R:
  (SEQ ID NO: 17)
AGTCAAGGAAGCCACTCAATTGATGAACGGGCTATTGATCATGTAATTAG
TTATGTCAC
GAL1-F:
  (SEQ ID NO: 18)
TGGCACTGGCCGTCGTTTTAACATGGCATTACCACCATATACATATCCAT
GAL1-R:
  (SEQ ID NO: 18)
AATTGTTAATATACCTCTATACTTTAACGTCAAGGAGAAAAAACTATAGG
TGAATCTTT
CYC1-F:
  (SEQ ID NO: 20)
ACTCAATTGATGAACGGGCTATTGATCATGTAATTAGTTATGTCACGCTT
ACATTCACG
CYC1-R:
(SEQ ID NO: 21)
CGTGCGTATTATCTCTTAACTCATAATGCCGGCCGCAAATTAAAGCCTTC
G
UP-F:
  (SEQ ID NO: 22)
TTGGTAACAGAAGATGGCAGTATTTCCA
UP-R:
(SEQ ID NO: 23)
GCGTAATCATGGTCATAGCTGTTTCCTGGGAGATGCGACGAATTACTGGC
LOXP-F:
(SEQ ID NO: 26)
GCCAGTAATTCGTCGCATCTCCCAGGAAACAGCTATGACCATGATTACGC
LOXP-R:
(SEQ ID NO: 27)
ATGGATATGTATATGGTGGTAATGCCATGTTAAAACGACGGCCAGTGCCA
DOWN-F:
(SEQ ID NO: 24)
GAAGGCTTTAATTTGCGGCCGGCATTATGAGTTAAGAGATAATACGCACG
DOWN-R:
  (SEQ ID NO: 25)
GAGAAAGGACTTAATCCGTACACAATGATT
YZ-TEV-F:
  (SEQ ID NO: 28)
AACAATGGGTTTCTGGCTGGAG
YZ-TEV-R:
  (SEQ ID NO: 29)
CCATCAATGGCTTCTAAAAGTTTTCAAAGAAGTG
```

### Example 5: Induction of Reversible Formation of Membraneless Organelle System RGG-x-(WGR-1)₂-EGFP

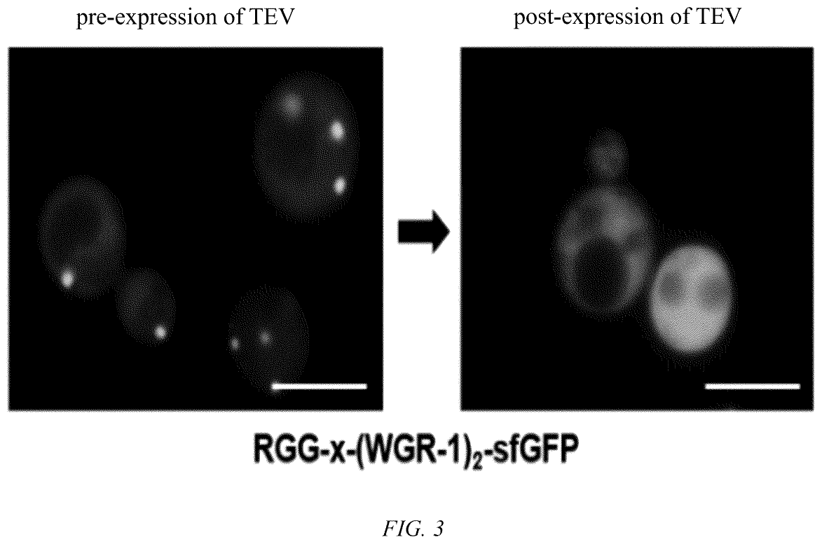

a) The single colony of the TEV-RW2-EGFP strain in Example 4 was picked up and cultured in a 2 mL of YPD liquid medium for 24 h. Then, as shown in FIG. 3, a membraneless organelle had been formed in the cell;
b) Galactose was added to the medium with a final concentration of 20 g/L to induce the expression of the TEV protease. After 24 h, the membraneless organelles disappeared, which reversed the phase separation of RGG-(WGR-1)₂, thus expanding the universality of RGG-(WGR-1)₂.

Obviously, the examples above are only examples for clear explanation, not limitation of the embodiments. For those of ordinary skill in the art, other changes or variations in different forms can be made on the basis of the above description. It is not necessary and impossible to exhaust all the embodiments here. The obvious changes or variations derived therefrom are still within the scope of protection created by the present invention.

1. A method for forming a membraneless organelle in a host cell, comprising steps of:
introducing a protein sequence containing an enzyme cleavage site for forming the membraneless organelle and a gene sequence encoding a cleavage enzyme into the host cell;
cultivating the host cell; and
cleaving the enzyme cleavage site on the protein sequence specifically by the cleavage enzyme encoded by the gene sequence,
wherein the protein sequence comprises SEQ ID NO: 2 or SEQ ID NO: 3.

2. The method according to claim 1, wherein the transcription of the gene sequence encoding the cleavage enzyme is initiated by a first promoter, and the transcription of the protein sequence containing the enzyme cleavage site for forming the membraneless organelle is initiated by a second promoter, and the first promoter is an inducible promoter and is different from the second promoter.

3. (canceled)

4. A recombinant protein for constructing a membraneless organelles, comprising an intrinsically disordered protein with an amino acid sequence as shown in SEQ ID NO: 1, and a short peptide located downstream of the intrinsically disordered protein, wherein the short peptide is a sequence as shown in SEQ ID NO.7 or two SEQ ID NO: 7 sequences connected in series.

5. A recombinant bacterium containing a membraneless organelle forming protein, comprising the recombinant protein of claim 4.

6. A system for constructing a reversible membraneless organelle, comprising at least two parts: a protein sequence containing an enzyme cleavage site for forming a membraneless organelle and a gene sequence for coding a cleavage enzyme, wherein
the protein sequence containing the enzyme cleavage site for forming the membraneless organelle comprises an intrinsically disordered protein with an amino acid sequence as shown in SEQ ID NO.1, a short peptide located downstream of the intrinsically disordered protein, and the enzyme cleavage site between the intrinsically disordered protein and the short peptide, wherein the short peptide is the sequence shown in SEQ ID NO.7 or two SEQ ID NO.7 sequences connected in series; and
the cleavage enzyme specifically cleaves the enzyme cleavage site.

7. A recombinant bacterium containing a reversible membraneless organelle, comprising the system of claim 6.

8. A method for producing a protein in living cells, comprising steps of: introducing the recombinant protein of claim 4 into the living cells.

9. Use of the recombinant bacterium of claim 5 in preparing cell drugs or protein drugs.
